(19) United States
(12) Patent Application Publication
(10) Pub. No.: US 2016/0136307 A1
(43) Pub. Date: May 19, 2016

(54) NANOPARTICLES FOR MAGNETIC PARTICLE IMAGING APPLICATIONS

(71) Applicant: Massachusetts Institute of Technology, Cambridge, MA (US)

(72) Inventors: He Wei, Cambridge, MA (US); Oliver T. Bruns, Boston, MA (US); Ou Chen, Mansfield, MA (US); Moungi G. Bawendi, Cambridge, MA (US)

(73) Assignee: MASSACHUSETTS INSTITUTE OF TECHNOLOGY, Cambridge, MA (US)

(21) Appl. No.: 14/941,307

(22) Filed: Nov. 13, 2015

Related U.S. Application Data
(60) Provisional application No. 62/080,236, filed on Nov. 14, 2014.

(51) Int. Cl. A61K 49/18; C01G 49/08

(52) U.S. Cl. CPC A61K 49/1818; C01G 49/08; A61K 49/186; A61K 49/183; C01P 2004/04; C01P 2002/72

(57) ABSTRACT

One method of preparing a nanoparticle can include decomposing a compound at a high temperature, adding an acid to the solvent to form a reaction mixture, increasing the temperature of the reaction mixture to boiling point of the reaction mixture, and heating the reaction mixture at the boiling point for 60 to 120 minutes to produce the nanoparticle. The coated nanoparticle or the nanoparticle can be used in magnetic particles imaging.

### PRIORITY CLAIM

This application claims priority to U.S. Provisional Application No. 62/080,236, filed on Nov. 14, 2014, which is incorporated by reference in its entirety.

### FIELD OF THE INVENTION

The invention relates to magnetic nanoparticles for imaging applications.

### STATEMENT REGARDING FEDERALLY SPONSORED RESEARCH OR DEVELOPMENT

This invention was made with government support under Grant Nos. CA126642 and CA119349 awarded by the National Institutes of Health, under Grant No. CHE-0714189 awarded by the National Science Foundation, and under Contract No. W911NF-07-D-0004 awarded by the U.S. Army Research Office. The government has certain rights in the invention.

### BACKGROUND

Nanometer sized particles often exhibit interesting electrical, optical, magnetic, and chemical properties, which cannot be achieved by their bulk counterparts. Magnetic nanoparticles can find applications in magnetic memory devices, ferrofluids, refrigeration systems, medical imaging, drug targeting, and catalysis. Magnetic oxide nanoparticles can be synthesized by using microemulsion and other methods.

### SUMMARY

In one aspect, a method of preparing a nanoparticle can include decomposing a compound at a temperature of 290° C.-390° C. in a solvent, adding an acid to the solvent to form a reaction mixture, increasing the temperature of the reaction mixture to boiling point of the reaction mixture, and heating the reaction mixture at the boiling point for 60 to 120 minutes to produce the nanoparticle.

In certain embodiments, the acid can include an oleic acid. The acid can include a stearic acid. The acid can include a nonadecanoic acid. The solvent can include a 1-hexadecene, a 1-octadecene, a 1-eicosene, a 1-dococene, or a 1-tetracosane, or a mixture thereof. The compound can include an iron oleate. The nanoparticle can include magnetite. The nanoparticle can have a size of between 15 and 35 nm.

In another aspect, a method for magnetic particle imaging can include introducing a nanoparticle that includes magnetite and has a size of between 15 and 35 nm into a subject; creating a magnetic particle imaging signal of the subject.

Other aspects, embodiments, and features will be apparent from the following description, the drawings, and the claims.

### BRIEF DESCRIPTION OF THE DRAWINGS

FIG. 1 shows TEM images of monodisperse iron oxide NPs with inorganic diameters of a) 11 nm, b) 12 nm, c) 15 nm, d) 18 nm, and e) 20 nm.

FIGS. 2A and 2B show a TEM image (FIG. 2A) and a superconducting quantum interference device (SQUID) curve (FIG. 2B) of as-synthesized 35 nm hydrophobic iron oxide NPs.

FIG. 3 shows an image of iron oxide/silica core/shell nanoparticles.

FIG. 4 shows the comparison of the 35 nm iron oxide nanoparticles and Resovist™.

FIG. 5A and FIG. 5B show X-ray powder diffraction (XRD) data.

### DETAILED DESCRIPTION

The development of biomedical imaging techniques has brought significant advances for diagnosis and therapy. Since most biological processes and diseases occur at the molecular and cellular levels, however, the precise real-time imaging and the understanding of these processes can be challenging.

Inorganic nanoparticles can have the potential for advancing imaging from the anatomy-based level to the molecular level, so-called “molecular imaging.” Upon conjugation with target-specific biomolecules, tiny probes (1-100 nm) can travel through the human body and they can identify the desired target by specific biological interactions, such as antibody-antigen, nucleic acid hybridization, and gene expression. Nanoparticles, such as iron oxide nanoparticles can be used in molecular imaging. Thermal-decomposition synthetic methods can be used for preparing MNPs and allow researchers to synthetically control the important features of these probes, such as size, magnetic dopants, magneto-crystalline phases, and surface.

Magnetic particle imaging (MPI) is an imaging technique that measures the magnetic fields generated by superparamagnetic nanoparticles (such as iron oxide) as tracers. MPI is an emerging medical imaging technique that can be a safer alternative to X-ray and CT using iodinated contrast agents, especially for patients with chronic kidney disease. Using various static and oscillating magnetic fields, and tracer materials made from such as iron oxide nanoparticles, MPI can perform background-free measurements of the particles' local concentration. The method can exploit the nonlinear remagnetization behavior of the particles and has the potential to surpass current methods for the detection of iron oxide in terms of sensitivity and spatiotemporal resolution.

MPI technology can be used to produce real-time images that accurately capture the activity in the cardiovascular system of a mouse. MPI allows determining the spatial distribution of magnetic nanoparticles, which can be used as tracers for medical imaging. The method provides a combination of features, which makes it a promising method for several clinical applications. It provides high spatial and temporal resolution, high sensitivity and is inherently quantitative. In contrast to several clinically used imaging methods, MPI is free of ionizing radiation and is thus considered to be safe even under long-term considerations. MPI scanners can be used to target special applications, for instance, cell tracking and interventional MPI.

MPI has near-perfect contrast because human (and most animals) tissue is diamagnetic, resulting zero MPI signal. MPI signals are therefore only from magnetic particles. MPI also has high sensitivity and resolution as 10⁻⁹ mol/L and 0.1-1 mm, respectively. Moreover, MPI is quantitative at any depth due to the fact that human (and most animals) tissue is transparent for low frequency magnetic field.

Nanoparticles can be prepared through the thermal decomposition of metal-complex precursors in hot non-hydrolytic organic solution containing surfactants. Thermal decomposition of the precursors can generate monomers and their aggregation above a supersaturation level can induce nucleation and subsequent nanoparticle growth. During these stages, it is possible to control the size, composition, and magneto-crystalline phase of NPs, by tuning growth parameters, such as monomer concentration, crystalline phase of the nuclei, choice of solvent and surfactants, growth temperature and time, and surface energy. Metal ferrite nanoparticles can be synthesized from precursors such as iron pentacarbonyl, iron cupferron, iron tris(2,4-pentadionate), and iron fatty acid complexes, in hot organic solvents containing fatty acids and amine surfactants. Nanoparticle size can be tuned within the range of 1 nm to approximately 150 nm.

For MPI, synthesis and size control of large magnetic nanoparticles are crucial (e. g., 15-35 nm) for increasing the MPI signal efficacy. For instances, in the currently used MPI contrast agent Resovist™, only less than 2% of the iron oxide nanoparticles actually contribute to the generated MPI signal. See, for example, Gleich, B.; Weizenecker, R. Nature 2005, 435, 1214; Goodwill, P. W.; Saritas, E. U.; Croft, L. R.; Kim, T. N.; Krishnan, K. M.; Schaffer, D. V.; Conolly, S. M. Advanced Materials 2012, 24, 3870, each of which is incorporated by reference in its entirety. By developing monodisperse and highly magnetic large iron oxide nanoparticles in our group, the number can be increased to greater than 90% of the nanoparticles in a sample contributing to the MPI signal.

A method of preparing a nanoparticle can include decomposing a compound at a temperature of 290° C.-390° C. in a solvent, adding an acid to the solvent to form a reaction mixture, increasing the temperature of the reaction mixture to boiling point of the reaction mixture, and heating the reaction mixture at the boiling point for 60 to 120 minutes to produce the nanoparticle.

A method for magnetic particle imaging can include using a nanoparticle that includes magnetite and can have an iron oxide inorganic diameter of between 15 and 35 nm, a hydrophilic silica shell having a thickness of between 5 and 10 nm, and can have a methoxy polyethylene glycol ligand coating.

### EXAMPLE

### Large Iron Oxide Nanoparticles

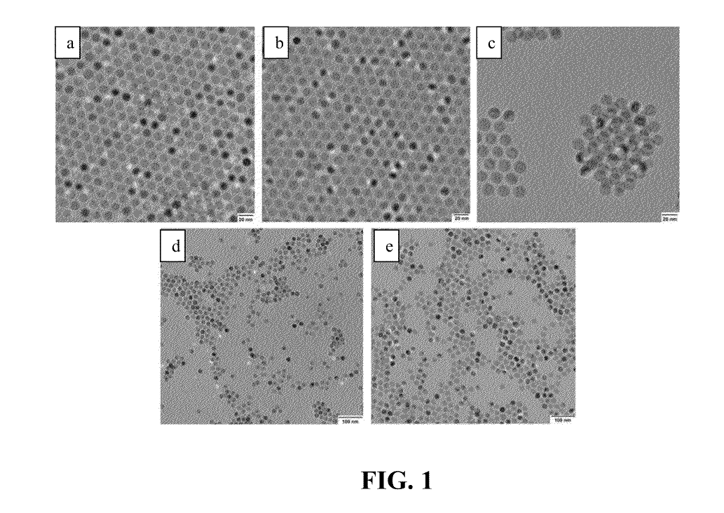

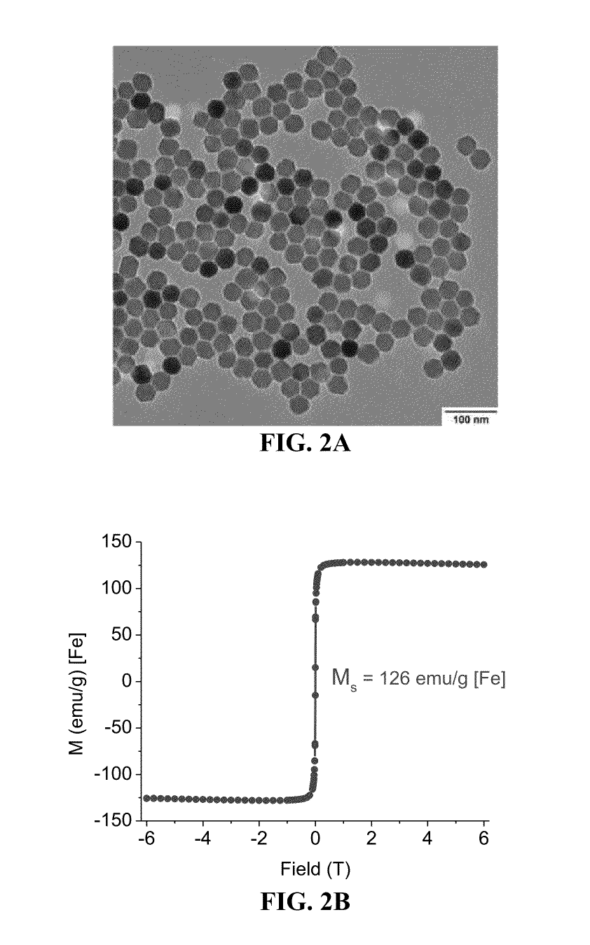

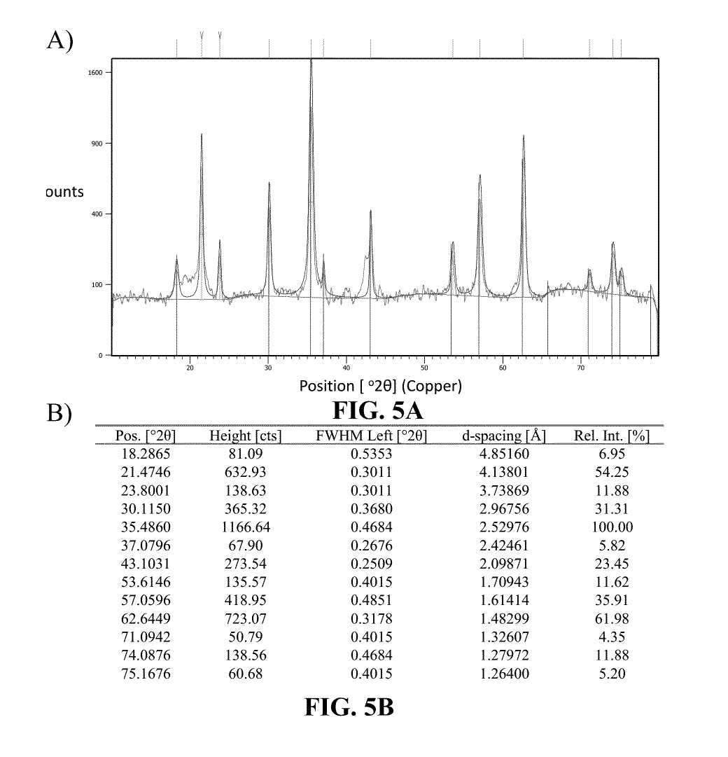

Magnetite (Fe₃O₄) magnetic NPs can be prepared through the decomposition of iron oleate precursors at high temperature (290° C.-390° C.). See, for example, Park, J.; An, K. J.; Hwang, Y. S.; Park, J. G.; Noh, H. J.; Kim, J. Y.; Park, J. H.; Hwang, N. M.; Hyeon, T. Nature Materials 2004, 3, 891, which is incorporated by reference in its entirety. As an example, 0.900 g ultra-dried iron oleate was added into 5.0 mL solvent. The solvent can be single specie or a mixture of 1-hexadecene, 1-octadecene, 1-eicosene, 1-dococene, and 1-tetracosane, depending on the reaction temperature to be used. Then 200-600 μL oleic acid organic ligands (or the same equivalent amount of stearic acid/nonadecanoic acid) were added into the above iron oleate solution. The temperature was increased at a rate of 30° C./min to the boiling point of the reaction mixture and it was held constant for 60-120 min. See, for example, Wei, H.; Insin, N.; Lee, J.; Han, H. S.; Cordero, J. M.; Liu, W. H.; Bawendi, M. G. Nano Letters 2012, 12, 22; Insin, N.; Tracy, J. B.; Lee, H.; Zimmer, J. P.; Westervelt, R. M.; Bawendi, M. G. Acs Nano 2008, 2, 197; Wei, H.; Bruns, O. T.; Chen, O.; Bawendi, M. G. Integrative Biology 2013, 5, 108, each of which is incorporated by reference in its entirety. Generally, higher reaction temperature and more organic ligands result in the formation of larger NPs. Upon completion, the reaction mixture was allowed to cool to room temperature, and the NPs were precipitated by adding chloroform and acetone. After centrifugation, the NPs were redispersed and stored in hexane while the supernatant was discarded. Transmission Electron Microscopy (TEM) images of as-synthesized iron oxide nanoparticles are shown in FIG. 1 and FIG. 2A, confirming their controlled and uniformed inorganic diameters. X-ray powder diffraction (XRD) data in FIG. 5A and FIG. 5B reveal that the as-synthesized 35 nm iron oxide nanoparticles are mainly magnetite (Fe₃O₄), with the first, second, and third strongest peaks at 35.5°, 62.6°, and 21.5°, respectively. Furthermore, FIG. 2B shows the superconducting quantum interference device (SQUID) measurements of 35 nm NPs. The iron concentration was determined by bathophenanthroline; see, for example, Wei, H.; Bruns, O. T.; Chen, O.; Bawendi, M. G. Integrative Biology 2013, 5, 108, which is incorporated by reference in its entirety. The saturation magnetization (Mₛ) of 35 nm NPs was found to be 126 emu/g [Fe], which is close to the value of bulk magnetite and consistent with the XRD data.

Iron Oxide Nanoparticles with a Silica Shell

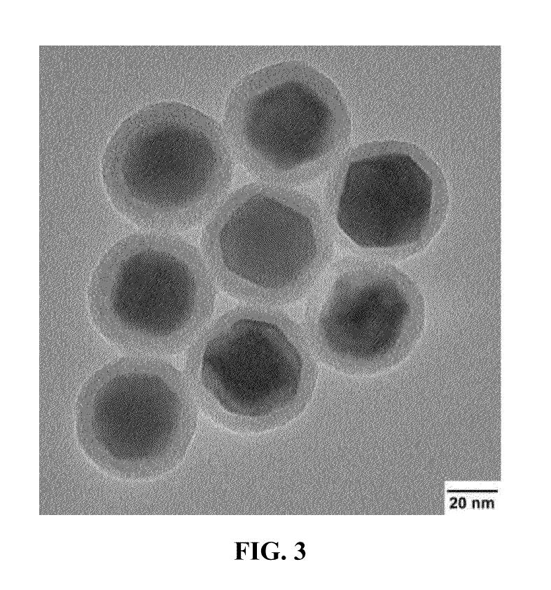

The water solubilization of ˜35 nm iron oxide nanoparticles can be achieved by the deposition of a hydrophilic silica shell. For example, the silica shell can be formed on the surface of iron oxide nanoparticles through the decomposition of tetraethyl orthosilicate (TEOS) in a cyclohexane solvent in the presence of Igepal® CO-520 and ammonium hydroxide at room temperature (RT) for ˜18 hours. See, for example, opovic, Z. et al., Angew Chem Int Edit 2010, 49 (46), 8649-8652, which is incorporated by reference in its entirety. The resulting iron oxide/silica core/shell nanoparticles have an inorganic diameter of ˜50 nm and they can be dispersed in water and phosphate buffered saline. FIG. 3 shows an image of iron oxide/silica core/shell nanoparticles.

In order to further improve the long-term stability of iron oxide/silica core/shell nanoparticles in aqueous media, they are coated by polyethylene glycol derivatives such as methoxy polyethylene glycol silane (mPEG-silane, M. W. ˜5000 g/mol) at 70° C. for 3 hours and RT for 18 hours.

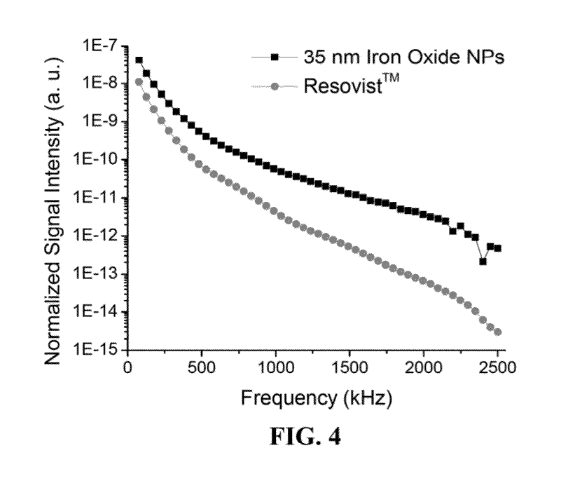

After the water-solubilization by mPEG-silane, the iron oxide nanoparticles were then tested by Magnetic Particle Spectroscopy (MPS) in order to quantitatively evaluate their potential MPI performance. As shown in FIG. 4, the performances of the 35 nm iron oxide nanoparticles and Resovist™ were compared using a MPS spectrometer operated at an excitation frequency of 25 kHz and an amplitude of 25 mT. It can be seen that the normalized signal intensity (by iron concentration) of the 35 nm iron oxide nanoparticles is about 10¹ higher than that of Resovist™ at low order harmonic frequencies (<500 kHz) and about 10² higher than that of Resovist™ at high order harmonic frequencies (>1500 kHz). The signal intensity at high order harmonic frequencies plays a more important role for MPI, and large signal intensity at high order harmonic frequencies usually results in better MPI performance. As a result, this result shows a potentially better MPI performance of the 35 nm iron oxide nanoparticles compared to that of the commercially available Resovist™, which is commonly used for MPI currently.

Other embodiments are within the scope of the following claims.

What is claimed is:

1. A method of preparing a nanoparticle comprising: decomposing a compound at a temperature of 290° C.-390° C. in a solvent, adding an acid to the solvent to form a reaction mixture, increasing the temperature of the reaction mixture to boiling point of the reaction mixture, and heating the reaction mixture at the boiling point for 60 to 120 minutes to produce the nanoparticle.

2. The method of claim 1, wherein the acid includes an oleic acid.

3. The method of claim 1, wherein the acid includes a stearic acid.

4. The method of claim 1, wherein the acid includes a nonadecanoic acid.

5. The method of claim 1, wherein the solvent includes a 1-hexadecene, a 1-octadecene, a 1-eicosene, a 1-dococene, or a 1-tetracosane, or a mixture thereof.

6. The method of claim 1, wherein the compound includes an iron oleate.

7. The method of claim 1, wherein the nanoparticle includes magnetite.

8. The method of claim 1, wherein the nanoparticle has a size of between 15 and 35 nm.

9. A method for magnetic particle imaging comprising:
introducing a nanoparticle that includes magnetite and has a size of between 15 and 35 nm into a subject; and
creating a magnetic particle imaging signal of the subject.

10. An imaging composition comprising:
a nanoparticle including magnetite and has a size of between 15 and 35 nm.

11. The imaging composition of claim 10, wherein the nanoparticle includes a silica shell.

12. The imaging composition of claim 10, wherein the nanoparticle includes a polyalkylene glycol coating.
